(19) United States
(12) Patent Application Publication
(10) Pub. No.: US 2017/0044497 A1
(43) Pub. Date: Feb. 16, 2017

(54) SELECTION AND USE OF STEM CELLS

(71) Applicant: National University of Ireland, Galway, Galway (IE)

(72) Inventors: Francis Peter BARRY, Galway (IE); Aline Myriam MORRISON, Galway (IE)

(73) Assignee: National University of Ireland, Galway, Galway (IE)

(21) Appl. No.: 15/118,561

(22) PCT Filed: Feb. 12, 2015
(86) PCT No.: PCT/EP2015/053023

§ 371 Date: Aug. 12, 2016

(30) Foreign Application Priority Data
Feb. 12, 2014 (EP) 14154923.8

(51) Int. Cl. C12N 5/0775; A61K 35/28

(52) U.S. Cl. CPC C12N 5/0663; A61K 35/28; A61K 2035/124

(57) ABSTRACT

A population of human stem cells is, or is selected to be, positive for CD271 and acetylated tubulin. Cells are isolated by a method comprising isolation of a cell from a mixed population of cells based on expression of cell surface markers, wherein the markers are CD271 and acetylated tubulin. The isolated cells may be used in therapy for example by producing tissues such as bone, cartilage or tendon, or in pharmaceutical compositions comprising isolated cells.

The present invention relates to methods of isolating stem cells, to stem cell populations obtained from the isolated cells and to uses of those populations and tissue derived therefrom.

Friedenstein et al. demonstrated that osteogenic potential, revealed by heterotopic transplantation of bone marrow (BM) cells, was associated with a minor subpopulation of BM-mono-nuclear cells (MNCs) (reviewed in Friedenstein, 1990). These MNCs were distinguishable from the majority of hematopoietic MNCs by their rapid adherence to plastic tissue culture vessels and by the fibroblast-like appearance of their progeny in culture, suggesting an origin from the stromal compartment of BM. As well as establishing BM stroma as the source, Friedenstein et al. provided a second breakthrough by showing that seeding of BM cell suspensions at clonal density results in the establishment of discrete colonies initiated by single cells (cells known as colony-forming unit-fibroblasts, CFU-F; Friedenstein et al., 1970).

Bone marrow (BM)-derived stem cells can be identified in a mixed population of plastic-adherent (PA), fibroblastic, MNCs that give rise to bone, fat or cartilage and secrete potent immunomodulatory and angiogenic proteins. Preclinical studies demonstrate that PA stem cells mediate potent immunomodulatory and angiopoietic responses in vivo. Currently, clinical trials are testing PA stem cells in 40 distinct degenerative, autoimmune and ischemic diseases.

In the human, approximately one in every 80,000 BM-MNC is a CFU-F forming cell. To date, the most simple and frequently used method of isolating these stem cells from BM is dependent upon adherence to tissue culture container plastic, according to which the MNC are left to incubate for 10-14 days and in the interim CFU-F will attach and form colonies at a recognised frequency (1:80,000). At 10-14 days these CFU-F are harvested by trypsin digest and replated in mesenchymal stem cell media at a density of 3-8000 CFU-Fs per cm². These CFU-Fs are then propagated in vitro until sufficient cell numbers are obtained to permit biochemical and cytological assessment. This approach is used widely but is regarded as inadequate for defining or purifying stem cells for clinical use, as only 1:80,000 BM-MNC plated are stem cells and the methods do not comply with good manufacturing protocols needed for clinical approval of related products.

Hence, in the prior art, stem cell populations have been identified based on an initial ability to adhere to a plastic surface. From this initial screen, cell populations are obtained as clonal populations from individual colony forming units on the surface. These have also been labelled in the literature “mesenchymal stem cells” though the term may be incorrect as non-mesenchymal stem cells may be included within the isolated cells. In a known isolation approach, these known cell populations are derived from stem cells that are positive for alkaline phosphatase and CD271.

Cell populations are prepared from these known isolated cells, such as by clonal expansion from a single, isolated cell, and used for transplantation. The results are variable, however, in that the transplanted cell populations sometime behave rather differently from batch to batch, and with an element of unpredictability. Transplanted cells may produce fat when bone- or collagen-forming cells are desired.

Prior art cell populations, as mentioned, tend to form bone, fat and cartilage, but with limited control, often making fat when bone or cartilage is required.

A further problem is that the starting cell population is essentially undefined, as isolation on the basis of adherence to plastic is not a sufficiently technical definition of a cell type.

Watson et al (Cells Tissues Organs, 2013, 197:496-504) relates to cells derived from human bone marrow and umbilical cord blood that are CD271 positive. US 2011/053183 relates to cells derived from human bone marrow that are CD271 positive, CD90 positive, CD45 negative and CD235a negative. WO 2013/117761 relates to cells derived from human bone marrow that are SDC positive, CD271^{bright} and CD45^{low}. Battula et al (Haematologica, 2009, 94:173-184) relates to cells derived from human bone marrow that are CD271 positive, and cells that are CD271^{bright} and CD45 positive or negative. WO 2013/151725 relates to three types of cells derived from skin that are: (i) CD146 positive; (ii) CD271 positive; or (iii) positive for SSEA3 and CD105. WO 2009/023566 relates to cells derived from bone marrow or peripheral blood that comprise cells that are CD271 positive.

However, these prior art cells have not been selected to be positive for acetylated tubulin as a cell surface marker.

Rojewski et al (Transfus. Med. Hemother., 2008, 35:168-184) reviews the phenotype of mesenchymal stems cells from various tissues, and confirms that a variety of cell markers have been analysed.

Wong et al (J Clin Invest., 2009, 119:336-48) relates to Clara cell secretory protein positive bone marrow cells that are positive for acetylated tubulin in an immunocytochemical study. However, the immunocytochemistry of Wong et al does not distinguish between a surface and/or cytosolic location of acetylated tubulin, which is more commonly considered to be a cytosolic cytoskeletal protein. Additionally, no cell sorting was performed on the basis of the present or absence of acetylated tubulin in Wong et al. In any case, the immunohistochemistry of Wong et al was done on cells that have undergone epithelial differentiation, and are thus not mesenchymal stems cells.

Thus, no cell populations, particularly stem cell populations, are known to be cell surface CD271 positive and acetylated tubulin positive.

It is an overall object of the invention to provide methods of isolations of stem cells and cell populations and tissues derived therefrom that are at least an alternative to the art, and an object of particular embodiments of the invention is to provide methods that are improved, for example through increased definition of the cells obtained, or cells that are improved, for example by increased reliability of their properties, rendering them more suitable for clinical applications.

The present invention is based upon prospective isolation of human stem cells, based on expression of markers or antigens that are expressed in a plurality of mammalian species. In methods and cell populations of the invention, cells are sorted on the basis of expression of a particular marker, this being referred to as prospective isolation, and then culture of the cells obtained, leaving to identification of cells, namely colony forming units (CFUs), which can be clonally expanded. The cell population obtained after the clonal expansion is then proposed for therapeutic, transplant and other uses.

According to a first aspect, the invention provides human stem cells selected to be positive for CD271 and acetylated tubulin.

“Positive for” CD271 or acetylated tubulin means that an identifiable marker or tag is associated with the cell, preferably located on the cell surface. For example, this marker may take the form of an antigen identifiable, e.g. by an antibody, on the cell surface.

The marker or tag may be used to identify or isolate the cell using manual or automated means such as single cell sorting, magnetic-activated cell sorting, or pulse cytophotometry (more commonly known as flow cytometry), preferably fluorescence-activated cell sorting (FACS).

The isolated stem cells may optionally be mesenchymal stem cells.

From cells that have been isolated, cell cultures and populations can be obtained. This can be achieved by clonal expansion of an isolated cell (e.g. a cell that is at least initially positive for both CD271 and acetylated tubulin) and then continued growth or culture of the cells obtained. Note that the cells obtained by this continued growth and culture and passaging tend initially to demonstrate the same marker spectrum as the originally isolated cell or cells. Over time the expression pattern may change. But the properties of the resultant population are linked to the criteria of the initial isolation (e.g. a cell that is at least initially positive for both CD271 and acetylated tubulin).

From cells that have been isolated, cell cultures and populations can generally be obtained having a high homogeneity, measured by expression of the markers or antigens used for the isolation. Hence, human stem cell populations are also provided by the invention expressing high levels of the first cell surface marker (one of CD271 and acetylated tubulin). The percentage of cells expressing the first marker may be 50% or more, 60% or more, 70% or more, 75% or more, 80% or more, or 90% or more. Independently, the cells also express high levels of the second marker (the other of CD271 and acetylated tubulin). The percentage of cells expressing the second marker may be 50% or more, 60% or more, 70% or more, 75% or more, 80% or more, or 90% or more.

In embodiments of the invention, 30% or more, preferably 50% or more, more preferably 70% or more, more preferably 80% or more and most preferably substantially all of the cells in the population are, or are selected to be, positive for both CD271 and acetylated tubulin.

This level of uniformity of the cell population makes them more suitable for use in therapy and research because they are a better characterised population of cells.

In embodiments requiring more precise characterisation of the cells, the cells may be isolated using further markers or tags. Accordingly, embodiments of the invention provide cell populations wherein the cells are additionally positive for one or more of STRO-1, STRO-3, W8B2, SDC2 and CD45.

The produced increased level of uniformity of the cell population makes them even more suitable for use in therapy and research because they are a better characterised population of cells.

Embodiments of the invention provide populations of stem cells that originate from bone marrow or are cells derived from pluripotent cells. This allows the cell population type to be suited to the particular research or therapeutic application.

Stem cells may be used to obtain tissues. Therefore, embodiments of the invention provide tissue obtained from a population of cells according to the invention. Preferably, the tissue obtained is bone, cartilage or tendon.

A further aspect of the invention provides a method of isolation of a human stem cell, comprising isolation of a cell from a mixed population of cells based on expression of cell surface markers, wherein the markers are CD271 and acetylated tubulin.

The mixed population of cells may be obtained from a source selected from bone marrow, adipose tissue, skeletal muscle, endometrium, placenta, umbilical cord blood, umbilical cord, Wharton's jelly, dental pulp and cells derived from pluripotent cells. Preferably, the source is bone marrow or cells derived from pluripotent cells.

In applications requiring more precise characterisation of the cells, the method may comprise isolating the cells on the basis of expression of one or more further cell marker or tag, in particular one or more surface marker. Accordingly, embodiments of the invention provide cell populations wherein the cells are additionally positive for one or more of STRO-1, STRO-3, W8B2, SDC2 and CD45.

Embodiments of the method of the invention are suitable for isolation of osteogenic cells, myogenic cells, cells giving rise to or forming ligaments or chondrogenic cells.

A further embodiment of the invention provides a method of obtaining a population of cells, comprising isolating cells according to the method of the invention described above, and deriving the population from those isolated cells.

Preferably, the method of obtaining a population of cells is a method of obtaining a clonal population of cells, comprising isolating a single cell according to the method of the invention described above, and deriving a clonal population of cells from the single cell.

An embodiment of the invention provides a method of obtaining a population of cells, comprising obtaining an initial population of cells according to the invention, or otherwise providing isolated cells positive for CD271 and acetylated tubulin, and then further growing and/or expanding and/or passaging the cells in culture.

An embodiment of the invention provides a method of obtaining a clonal population of cells, comprising isolating a single cell according to invention, or otherwise providing a single cell positive for CD271 and acetylated tubulin, and deriving a clonal population of cells from the single cell.

The invention provides a population of cells obtainable according to the method of the invention.

A particular embodiment of the invention provides a population of cells, obtained by:
providing a human stem cell;
deriving a clonal population of cells from the human stem cell; and
optionally, further growing and/or expanding and/or passaging the cells in culture,
wherein the human stem cell (i) is isolated from bone marrow, and (ii) is positive for both CD271 and acetylated tubulin.

Tissues are provided by the invention, by obtaining cells according to described methods, and obtaining tissue therefrom. Tissue selected from bone, cartilage, muscle, fat cells and tendon can be obtained in this way. Tissue for reconstructive surgery can thus be obtained.

A further use of the invention lies in providing cells for and assays using the isolated cells and progeny thereof. Hence, a method of conducting an assay comprises obtaining cells according to the described methods, or otherwise providing an isolated cell positive for CD271 and acetylated tubulin, and using those cells in the assay.

In addition, CD271 and acetylated tubulin positive cells and cell populations are derived in specific methods of the invention from starting cells that are more highly defined than in the prior art, by reference to a marker that persists in cells and progeny. In itself, this is an advantage. The cell population is an acceptably defined population.

Cells and tissues of the invention, and compositions comprising the cells and tissues, can be used to treat various human conditions and diseases, including in particular those treatable using cells and products derived from existing stem cell products. The cells and tissue may interact with dendritic cells and drive IFN-β secretion, and hence may be used as a tumor suppressor. Cancers in general may be treated using the invention, specifically including hepatocellular carcinoma, cervical cancer, pancreatic cancer, prostate cancer, fibrosarcoma, medullablastoma, and astrocytoma. Lung diseases may be treated, including: acute lung injury (ALI); acute respiratory distress syndrome (ARDS); chronic obstructive pulmonary disosorder (COPD); and idiopathic pulmonary fibrosis (IPF). The cells and tissues may be used to treat sepsis and sepsis-induced multiorgan failure, bone marrow transplant (BMT) or haematopoietic stem cell (HSC) rejection; solid organ transplant (SOT) rejection (including liver, kidney, skin, cornea, heart, lung); acute toxin-induced liver failure; autoimmune hepatitis; primary biliary cirrhosis (PBC) and primary sclerosing cholangitis (PSC); osteonecrosis; degenerative disc disease; rheumatoid arthritis; osteoarthritis and delayed bone healing in diabetic patients; autoimmune nephritis including Wegener's granulomatosis (WG); burns such as severe burns; muscle wasting conditions and atrophic syndromes including sarcopenia; cachexia and other muscle wasting conditions including the muscular dystrophies (Duchenne and Becker); congestive heart failure, acute myocardial infarction and stroke; type 1 diabetes; type 2 diabetes; diabetic retinopathy and other retinopathies; diabetic nephropathy and other nephropathies; diabetic neuropathy and other neuropathies; non-healing diabetic ulcers; diabetic cardiomyopathy and other myopathies; athersclerosis; peripheral artery disease and critical limb ischemia; uveitis; (wet or dry) acute macular degeneration (AMD); retinal and corneal damage; autoimmune conditions such as autoimmune gastritis (AIG); graft-versus-host disease (GVHD); multiple sclerosis and demyelinating diseases; thyroid disease; inflammatory bowel diseases including Crohns Disease, ulcerative colitis and fistulising Crohn's Disease; scleroderma; lupus (e.g. systemic lupus erythematosus—SLE); Graves' Disease; and autoimmune lymphoproliferative disease (ALPS).

Also provided by the present invention is a pharmaceutical composition for treating a disease or disorder in a human. The pharmaceutical composition suitably comprises cells or tissue of the invention in an amount effective to treat the disease or disorder. The cells may thus be administered with an acceptable pharmaceutical carrier. For example, the cells may be administered as a cell suspension in a pharmaceutically acceptable liquid medium for injection. Examples of liquid medium are saline, phosphate buffered saline, optionally also containing additional materials such as dimethylsufoxide (DMSO) and human serum albumin. The cells and tissue may generally be administered in a variety of formats as known for existing stem cell and like products and tissue derived therefrom. They can be administered systemically, e.g. by intravenous infusion, or direct injection. The compositions may comprise a matrix or scaffold, or the cells or tissue may be administered by injection into a site already comprising matrix or scaffold in situ. The cells or tissue may thus be administered in combination with hyaluronic acid, collagen or other extracellular matrix. Further formulation and administration examples that can be applied mutatis mutandis to the cells and tissue of the invention may be found in the art, e.g. in WO2001080865, EP2545928 and WO1999061587. A method of treatment of a human is provided, comprising administering to the human a composition of the invention. Cells or tissue according to the invention are provided for use in treatment of a disease or disorder of a human. Embodiments of the methods and uses comprise embodiments generally of the invention as described herein.

Suitable antibodies are available to the skilled person for performing sorting and isolation based on the identified markers. Human CD271 and acetylated tubulin antibodies are commercially available and will be known to the skilled person. For example, a suitable acetylated tubulin antibody may be obtained from Sigma-Aldrich under catalogue number T-7451.

The invention is now described in specific embodiments with reference to the accompanying drawings, in which:

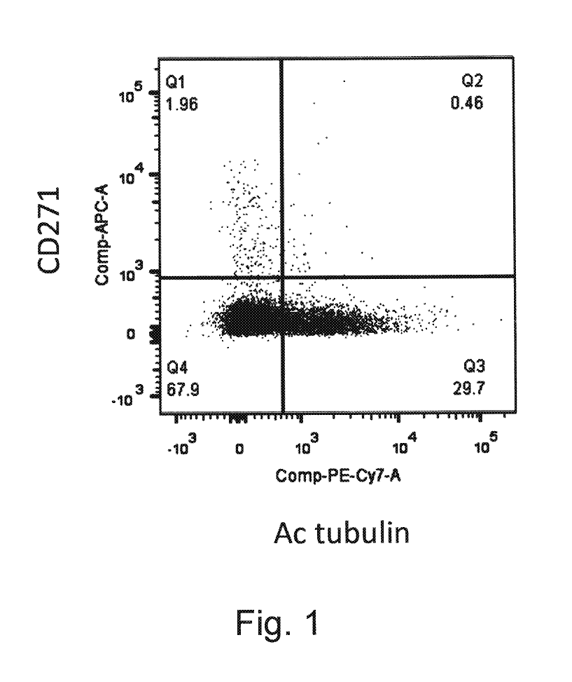

FIG. 1 shows a FACS profile of acetylated tubulin versus CD271 for cells derived from human bone marrow;

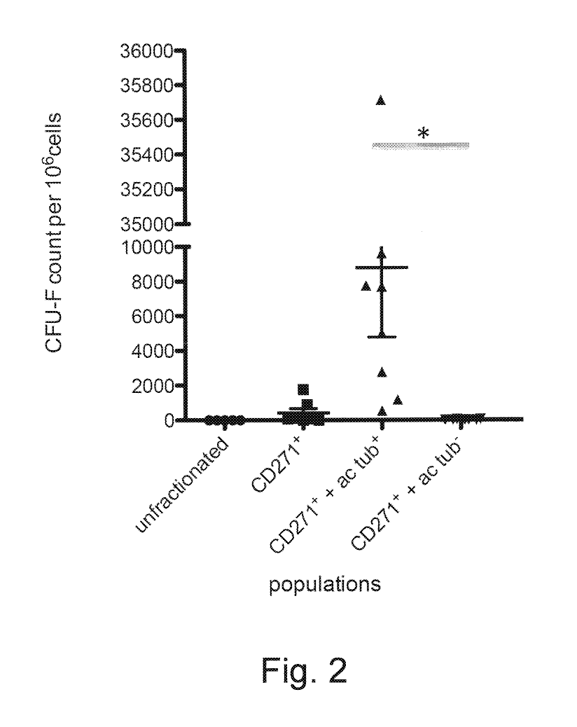

FIG. 2 shows the frequency of CFU-F formation in cells that are positive for CD271 and acetylated tubulin (i.e. cells from top right quadrant of FIG. 1);

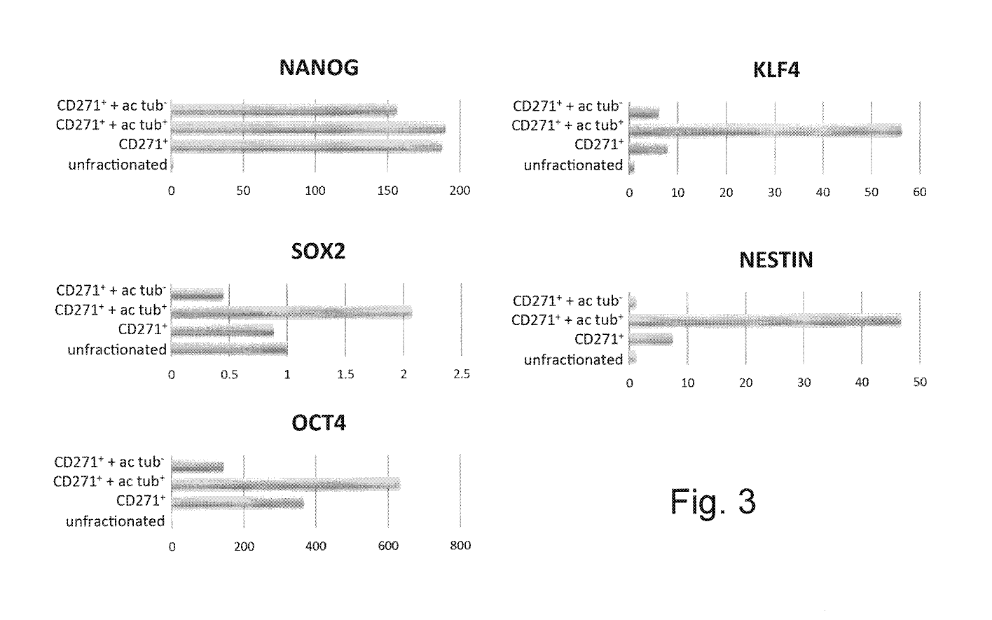

FIG. 3 shows expression of stemness genes measured by relative qPCR in cells that are positive for CD271 and acetylated tubulin;

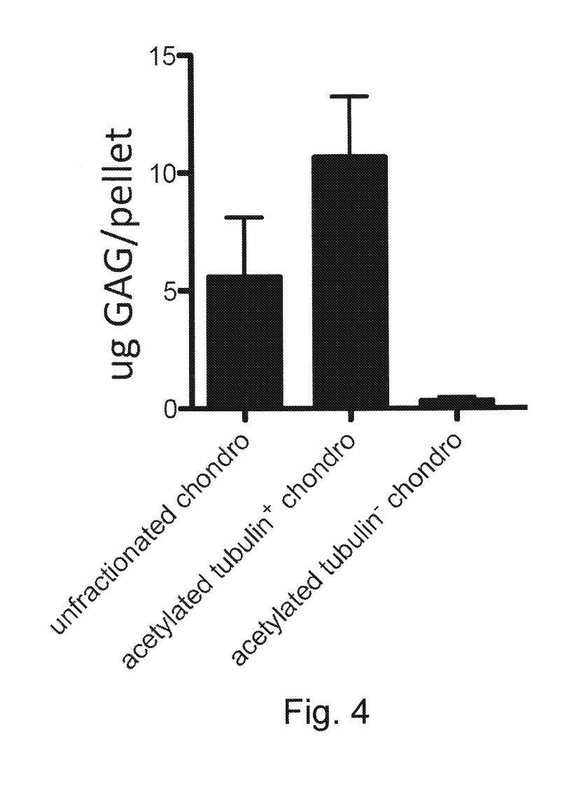

FIG. 4 shows the level of chondrogenesis in cells that are positive for CD271 and acetylated tubulin;

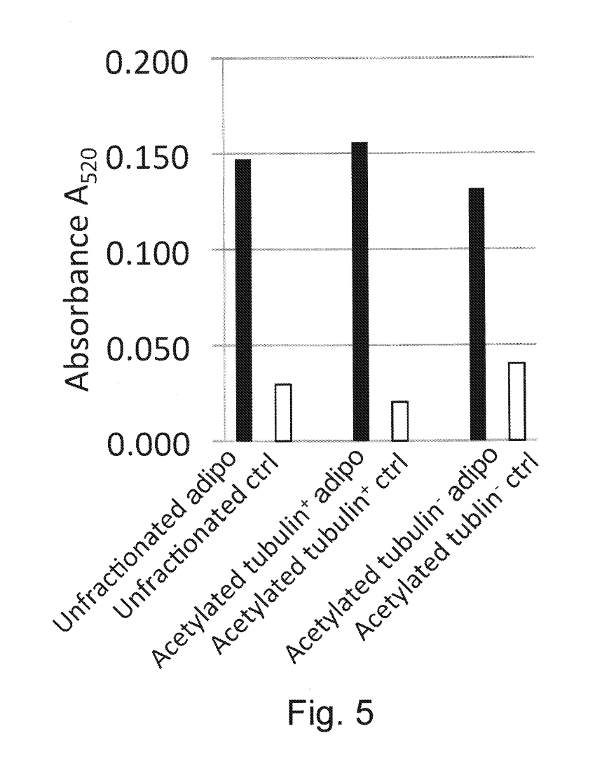

FIG. 5 shows the level of adipogenesis in cells that are positive for CD271 and acetylated tubulin; and

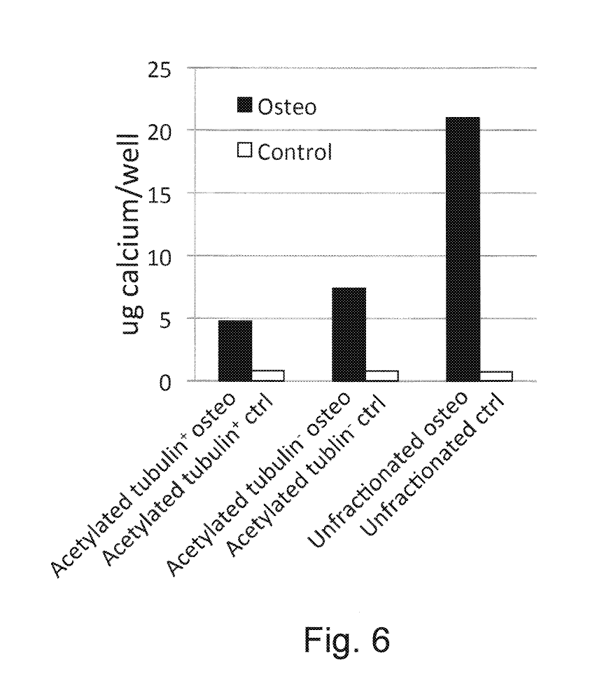

FIG. 6 shows the level of osteogenesis in cells that are positive for CD271 and acetylated tubulin.

### EXAMPLE 1

### Isolation of Stem Cells Positive for CD271 and Acetylated Tubulin using FACS

Bone marrow was obtained and diluted 1:1 with phosphate buffered saline (PBS) and mononuclear cells (MNC) were isolated using Ficoll-Plus according to a standard protocol.

The obtained MNC were then stained with anti-CD45, glycophorin A, CD271 and acetylated tubulin antibodies and sorted by FACS cell sorting for a cell population that was positive for CD271, acetylated tubulin, CD45 (mid level gate) and negative for glycophorin A.

Cells that were both CD271 positive and acetylated tubulin positive isolated using FACS formed 5,000 fibroblastic colonies (CFU-Fs) per million cells, whereas cells isolated by plastic adherence formed 7 colonies per million cells and cells that were sorted on the basis of being positive for CD271 but negative for acetylated tubulin formed 59 colonies per million cells.

As the cell sorting was performed on non-permeabilised cells, the CD271 and acetylated tubulin markers were present on the surface of the cells and the cells were viable.

### EXAMPLE 2

Isolation of Stem Cells Positive for CD271 and Acetylated Tubulin using pluriBeads/Dynal Isolation

Bone marrow was obtained, diluted 1:1 with phosphate buffered saline (PBS) and labelled with pluriSelect CD271 pluriBeads according to the manufacturer's protocol in which the bone marrow was incubated with the CD271 pluriBeads for 30 minutes while rotating at room temperature. Then the bone marrow was passaged over a strainer and the positive held-back fraction (CD271 positive cells) was detached from the pluriBeads.

The obtained CD271 positive cells were then labelled with an acetylated tubulin antibody for 20 minutes at 4° C., then washed to remove unbound antibody. The cells were then labelled with anti-mouse Ig Dynal beads for 20-30 minutes while rotating at 4° C. The acetylated tubulin positive cells were then separated from the negative cells using an appropriate magnet.

A FACS profile of acetylated tubulin versus CD271 shows that a minority of the cells express both markers (FIG. 1).

Cells that were both CD271 positive and acetylated tubulin positive isolated using pluriBeads/Dynal formed 584 fibroblastic colonies (CFU-Fs) per million cells, whereas cells that were sorted on the basis of being CD271 positive but negative for acetylated tubulin formed 19 colonies per million.

On average, cells selected to be both CD271 positive and acetylated tubulin positive (“CD271⁺+ac tub⁺”) formed more colonies than “unfractionated” cells (i.e. cells that were not subject to selection in respect of CD271 and acetylated tubulin), cells selected to be CD271 positive but unselected for acetylated tubulin (“CD271⁺”), or cells selected to be CD271 positive but acetylated tubulin negative (“CD271⁺+ac tub⁻”)—see FIG. 2. By way of example, the difference between “CD271⁺+ac tub” cells and “CD271⁺+ac tub⁻” cells was significant with a p value of less than 0.0001 using a one-way ANOVA.

As the cell sorting was performed on non-permeabilised cells, the CD271 and acetylated tubulin markers were present on the surface of the cells and the cells were viable.

### EXAMPLE 3

### Isolation of Stem Cells Positive for CD271 and Acetylated Tubulin Using pluriBeads

Bone marrow was obtained, diluted 1:1 with phosphate buffered saline (PBS) and labelled with pluriSelect CD271 pluriBeads according to the manufacturer's protocol in which the bone marrow was incubated with the CD271 pluriBeads for 30 minutes while rotating at room temperature. Then the bone marrow was passaged over a strainer and the positive held-back fraction (CD271 positive cells) was detached from the pluriBeads.

pluriBeads were then coated with the acetylated tubulin antibody and used for a second isolation step over the next strainer and the positive held-back fraction (CD271 positive and acetylated tubulin positive cells) was detached from the pluriBeads.

Cells that were both CD271 positive and acetylated tubulin positive formed 7,692 fibroblastic colonies (CFU-Fs) per million cells, whereas cells that were sorted on the basis of being CD271 positive formed 1,766 colonies per million cells, and cells that were sorted on the basis of being CD271 positive but negative for acetylated tubulin formed no colonies.

Accordingly, significant increased CFU-F properties were consistently seen in the cell populations positive for both markers in all experiments.

As the cell selection was performed on non-permeabilised cells, the CD271 and acetylated tubulin markers were present on the surface of the cells and the cells were viable.

### EXAMPLE 4

### Stemness Gene Expression

Cells selected to be CD271 positive and acetylated tubulin positive were isolated according to Example 2. The expression of stemness genes in these cells was analysed and compared to three other groups of cells using relative qPCR analysis of RNA according to standard protocols using a SensiFAST SYBR Hi-ROX kit (Bioline) and a StepOnePlus thermal cycler (Applied Biosystems). The obtained RNA levels were normalised to the levels of GAPDH and DROSHA reference RNA.

The expression pattern for NANOG, KLF4, SOX2, nestin and OCT4 for cells selected to be CD271 positive and acetylated tubulin positive (“CD271⁺+ac tub⁺”) was different compared to “unfractionated” cells, cells selected to be CD271 positive but unselected in respect of acetylated tubulin (“CD271⁺”) and cells selected to be CD271 positive but acetylated tubulin negative (“CD271⁺+ac tub⁺”)—see FIG. 3. The expression levels of 4 out of 5 stemness genes was higher in the doubly selected population.

### EXAMPLE 5

Chondrogenesis, Adipogenesis and Osteogenesis of Stem Cells Positive for CD271 and Acetylated Tubulin

Cells were isolated according to Example 2, and functional characterisation of the resulting cells was carried out by testing their chondrogenic, adipogenic and osteogenic capacity according to the methods described in Fábián et al, Stem Cell Res., 2014, 12:646-58.

Following chondrogenic induction, cultured cells that were both CD271 positive and acetylated tubulin positive produced twice as much glycosaminoglycans (GAG) than unfractionated cells (FIG. 4). Additionally, cells that were CD271 positive but acetylated tubulin negative produced insignificant levels of GAG (FIG. 4). Using the markers of the invention thus segregated a population capable of chondrogenesis from cells that were not.

Following adipogenic induction, cultured cells that were both CD271 positive and acetylated tubulin positive were just as able to engage in adipogenesis as unfractionated cells or cells that were CD271 positive but acetylated tubulin negative, as shown by absorbance of light, which is proportionate to the amount of lipid production (FIG. 5). In this assay, control (“ctrl”) cells that were cultured in maintenance media, not adipogenic induction media, produced no or very low lipid compared with differentiated cells (FIG. 5): the background level is from the absorbance from the isopropanol used in the assay.

Following osteogenic induction, we measured the amount of calcium accumulation in cultured cells that were both CD271 positive and acetylated tubulin positive, in unfractionated cells and in cells that were CD271 positive but acetylated tubulin negative (FIG. 6). Control (“ctrl”) cells that were cultured in maintenance media, not osteogenic induction media, accumulated less calcium than differentiated cells regardless of the surface marker selection protocol used (FIG. 6).

Thus, stem cells selected for surface expression of CD271 and acetylated tubulin preferentially favoured chondrogenesis.

The invention hence provides cells selected to be positive for CD271 and acetylated tubulin, methods of obtaining the same, uses thereof and tissues derived therefrom.

1. A population of human stem cells selected to be positive for CD271 and acetylated tubulin.

2. The population of cells of claim 1, wherein 30% or more of the cells are positive for both CD271 and acetylated tubulin.

3. The population of cells of claim 2, wherein 50% or more of the cells are positive for both CD271 and acetylated tubulin.

4. The population of cells of claim 2, wherein 70% or more of the cells are positive for both CD271 and acetylated tubulin.

5. The population of cells according to of claim 2, wherein substantially all of the cells are positive for both CD271 and acetylated tubulin.

6. Tissue obtained from the population of cells of claim 2.

7. A method of isolation of a human stem cell, comprising isolation of a cell from a mixed population of cells based on expression of cell surface markers, wherein the markers are CD271 and acetylated tubulin.

8. The method of claim 7, wherein the mixed population of cells is obtained from a source selected from bone marrow, adipose tissue, skeletal muscle, endometrium, placenta, umbilical cord blood, umbilical cord, Wharton's jelly, dental pulp and cells derived from pluripotent cells.

9. A method of obtaining a clonal population of cells, comprising (i) isolating a single cell according to the method of claim 7, or (ii) providing a single cell positive for CD271 and acetylated tubulin, and (iii) deriving a clonal population of cells from the single cell of (i) or (ii).

10. The method of claim 7, further comprising growing and/or expanding and/or passaging the human stem cell in culture.

11. The population of cells of claim 1, obtained by:
providing a human stem cell;
deriving a clonal population of cells from the human stem cell; and
optionally, further growing and/or expanding and/or passaging the cells in culture,
wherein the human stem cell (i) is isolated from bone marrow, and (ii) is positive for both CD271 and acetylated tubulin.

12-13. (canceled)

14. A method of conducting an assay, comprising obtaining a cell according to the method of claim 7, or providing an isolated cell positive for CD271 and acetylated tubulin, and using the cell in the assay.

15. A method of treatment of a disease or disorder of a human, comprising administering to the human an effective amount of the cell of claim 1.
